(19) United States
(12) Patent Application Publication
(10) Pub. No.: US 2025/0027037 A1
(43) Pub. Date: Jan. 23, 2025

(54) CELL CULTURE SUBSTRATE COMPRISING NONWOVEN MAT CONFIGURED FROM BIOCOMPATIBLE RESIN FIBERS, AND METHOD FOR PRODUCING SAME

(71) Applicant: ORTHOREBIRTH CO., LTD., Yokohama-shi, Kanagawa (JP)

(72) Inventors: Masahiro Kumano, Kanagawa (JP); Fumihide Bunai, Kanagawa (JP)

(73) Assignee: ORTHOREBIRTH CO., LTD., Yokohama-shi, Kanagawa (JP)

(21) Appl. No.: 18/715,494

(22) PCT Filed: Nov. 28, 2022
(86) PCT No.: PCT/JP2022/043735

§ 371 Date: May 31, 2024

Related U.S. Application Data
(60) Provisional application No. 63/284,638, filed on Dec. 1, 2021.

(51) Int. Cl. C12N 5/00; C12N 5/0775

(52) U.S. Cl. CPC C12N 5/0068; C12N 5/0031; C12N 5/0662; C12N 2533/18; C12N 2533/50

(57) ABSTRACT

Disclosed is a cell culture substrate including a non-woven mat formed of biocompatible fibers produced by an electrospinning method. The biocompatible fibers have a fiber length of 2 mm to 80 mm and a fiber diameter of 10 to 80 μm, and contain 20 to 50 vol % (about 45 to 75 wt %) inorganic filler particles and 50 to 80 vol % (about 25 to 55 wt %) biocompatible resin. The inorganic filler particles are partially exposed on the fiber surface to form an uneven structure. The non-woven mat can trap the seeded mesenchymal stem cells between the fibers and attach them to the fibers. A plurality of short fibers forming the non-woven mat are entangled with each other and adhered and connected at multiple points of contact, forming a three-dimensional structure in which a microenvironment in which cells can adhere and grow in the space between the biocompatible fibers and fibers.

### TECHNICAL FIELD

Present invention relates to a non-woven mat used as a cell culture substrate for growing mesenchymal stem cells (MSCs), and a method for producing the cell culture substrate.

### BACKGROUND TECHNOLOGY

Cell culture is a process of taking cells out of a living organism and keeping them alive. In order to culture cells in vitro, it is necessary to create an environment in vitro that is similar to the microenvironment in vivo.

Recently, non-woven fabrics made of nanofibers produced by using electrospinning method are proposed as a cell culture substrate. Non-woven fabrics made of fibers produced by electrospinning method form a three-dimensional structure that is similar to the extracellular matrix (ECM) and can be used as an excellent three-dimensional cell culture substrate in place of conventional two-dimensional cell culture plates.

Non-woven fabrics used as three-dimensional cell culture substrates must have spaces between fibers where cells can enter and nutrients and oxygen contained in the culture medium can be supplied therein. However, fibers produced by electrospinning method are generally extremely thin, with fiber diameters ranging from several tens of nm to several μm, so the fibers are densely entangled with each other and do not form sufficient gaps between fibers. Therefore, measures have been proposed to secure gaps between fibers (Enhancing cell infiltration of electrospun fibrous scaffolds in tissue regeneration, Jinglei Wu et al. University of TEXAS Bioactive Materials 1 (2016) 56-64).

In order to attach MSCs, which are adhesive cells, to a scaffold, the surface of the scaffold must be covered with adhesive proteins so that cells can attach to the substrate having a protein layer on its surface. Therefore, in order to use non-woven fabrics as cell culture substrates, it is important that surfaces of the fibers that form the non-woven fabrics effectively adsorb the adhesive proteins contained in the culture medium.

It has also been reported that cells can be attached to a scaffold with an uneven surface shape more easily than to those with a planar surface. In this case, if the unevenness is extremely fine, cells cannot recognize the unevenness and it is no different from a planar surface. On the other hand, if the unevenness is too high, cells cannot attach to the scaffold beyond the unevenness.

Furthermore, it has been reported that when a non-woven fabric is used as a cell culture substrate, cells tend to attach at intersections of the fibers and migrate along the fiber alignment from there (Mechanical tensile strengths and cell proliferative Mechanical tensile strengths and cell proliferative activities of electrospun poly(lactic-co-Glycolic acid) composites containing β-tricalcium phosphate Phosphorous Research Bulletin Vol. 26 Special Issue (2012) pp 109-112 Shingo Ito et al).

### PRIOR ART REFERENCES

### Patent Document

Patent document 1: U.S. Pat. No. 6,639,035 Patent Gazette

### Non-Patent Document

Non-patent literature 1: Enhancing cell infiltration of electrospun fibrous scaffolds in tissue regeneration, Jinglei Wu et al. University of TEXAS Bioactive Materials 1 (2016) 56-64
Non-Patent Document 2: Mechanical tensile strengths and cell proliferative activities of electrospun poly(lactic-co-Glycolic acid) composites containing Phosphorous Research Bulletin Vol. 26 Special Issue (2012) pp 109-112 Shingo Ito et al.

### SUMMARY OF INVENTION

### Problem to be Solved

In order for cells to proliferate and differentiate in vitro, the cell culture substrate must provide the same environment for cells as in vivo. However, there are a wide range of requirements for cell culture substrates. For this reason, although use of a non-woven fabric as a cell culture substrate has been proposed, its development has not been easy in practice. As a result, cell culture substrates using non-woven fabrics have not been successfully commercialized.

### Measure to Solve the Problem

In order to solve above problem, inventors of the present invention made an intensive study and reached the idea of utilizing ReBOSSIS (registered trademark), which is a cotton-like artificial bone previously developed and commercialized by the applicant, for a cell culture substrate. The above cotton-like artificial bone is a bone regeneration material for implantation in a bone defect and has received a high reputation in the market with many clinical results. The cotton-like structure is composed of numerous fibers with diameters of several tens of micrometers, and the spaces between the fibers form the microenvironment necessary for entry, proliferation, and differentiation of osteoblastic cells. In addition, ReBOSSIS contains a large amount of calcium phosphate particles in the fibers by using a kneading method, and the large amount of particles are partially exposed on the surface of the fibers to form a concave-convex shape that is suitable for cell attachment. Inventors reached utilizing this unique structure of ReBOSSIS for a three-dimensional cell culture substrate in vitro for MSC.

Based on the above idea, inventors made a further study and succeeded to modify the production process of ReBOSSIS such that spinning solution emitted from the nozzle are distributed and deposited on the rotating drum collector as many short fibers to produce a non-woven mat with a three-dimensional structure composed of biocompatible fibers. By conducting a culturing experiment by seeding MSCs onto the non-woven mats thus prepared, it was found that the seeded MSCs are effectively trapped by the non-woven mats, attached to the fibers, and shows a high proliferation.

Based on above findings, inventors of present invention have reached a cell culture substrate comprising a non-woven mat made of biocompatible fibers produced by using an electrospinning method,
the biocompatible fibers contain 20 to 50 vol % (about 45 to 75 wt %) of inorganic filler particles and 50 to 80 vol % (about 25 to 55 wt %) of biocompatible resin,
a surface of the biocompatible fiber has an uneven structure formed by having inorganic filler particles partially exposed thereon,
the non-woven mat has a three-dimensional structure having a microenvironment in which a plurality of fibers with a fiber length of 2 mm to 80 mm and a fiber diameter of 10 to 80 μm are entangled each other and adhered and connected at multiple points of contact so that seeded cells are trapped in the non-woven mat, allowing cells to attach to the fiber and grow in the spaces between the fibers of the biocompatible fiber mat.

Further, inventors of present invention have reached a method for manufacturing a cell culture substrate comprising a non-woven mat made of biocompatible fibers produced by electrospinning method, the method comprising:
preparing a composite containing 20-50 vol % (approx. 45-75 wt %) of inorganic filler particles and 50-80 vol % (approx. 25-55 wt %) of biocompatible resin by using a kneading process, and dissolving the composite in a solvent to produce a spinning solution with a resin concentration of 8-12% by weight,
filling the spinning solution into a syringe of the electrospinning apparatus and extruding the spinning solution at a predetermined feed rate from a discharge port of a nozzle with an inner diameter of 0.7 mm to 0.9 mm installed downward at the top of the electrospinning apparatus,
applying a predetermined voltage to the nozzle so that an electric field is generated between the nozzle and a rotating drum collector installed at a distance of 180 mm to 240 mm from the nozzle and electrically grounded and the spinning solution is ejected from the nozzle in a form of fibers by Taylor cone phenomenon that is caused by charging the spinning solution,
depositing the fibers emitted from the nozzle on a rotating drum collector by falling down the fibers while spirally swinging due to a destabilization phenomenon of a flight trajectory of fibers in an electric field, wherein the fibers are torn apart to become a plurality of curved short fibers of 2 mm to 80 mm in length before reaching the rotating drum collector, and the plurality of curbed short fibers are distributed and deposited on the rotating drum as the nozzle slides horizontally,
wherein the deposited plurality of curved short fibers are entangled each other on the rotating drum collector such that the fibers are adhered and connected each other at points of contact to form a non-woven mat having a microenvironment therein so that the seeded cells are trapped in the mat to adhere to the fibers and grow in the space between the biocompatible fibers, and
collecting the non-woven mat from the rotating drum collector and cutting into a desired dimension as a cell culture substrate.

Preferably, the fibers of the non-woven mat contain 20-30 vol % (about 45-55 wt %) of inorganic filler particles, the fibers are 20 mm-80 mm in length, and a mat shape is formed by the plurality of curved fibers entangled in random directions and adhered each other.

Preferably, the fibers constituting the non-woven mat contain 40-50 vol % (about 65-75 wt %) inorganic filler particles, the fibers are 2 mm-10 mm in length, and the mat shape is formed by a plurality of short curved fibers entangled in random directions and adhered to each other.

Preferably, the inorganic filler particles are HAp particles, and more preferably needle-shaped HAp particles.

Preferably, particle size of the inorganic filler particles is 1 to 5 μm.

Preferably, the non-woven mat is cut to fit the dimension of the cell culture well plate or dish.

Preferably, the non-woven mat has a thickness of 0.1 mm to 0.5 mm.

Preferably, the outer diameter of the biocompatible fibers that form the non-woven mat is 10 to 80 μm, more preferably 10 to 60 μm.

Preferably, the resin fibers comprise PLGA, PLA or PCL.

Preferably, the fibers comprising the non-woven mat are three-dimensionally entangled with a number of fibers with an inter-fiber distance of 10-200 μm, so that mesenchymal stem cells of approximately 10 μm in diameter can penetrate into the spaces between the fibers and be retained therein.

### Advantage of the Invention

The non-woven mats produced by an embodiment of the method of the invention have an outer diameter of 10 to 80 μm (see FIGS. 4(a) (b) and 5(a) (b)), allowing cells to easily enter into the microenvironment formed in the three-dimensional structure of the non-woven mat so that the cells can attach to the fiber and proliferate in a trapped state.

The non-woven mat produced by the method of an embodiment of the invention have numerous filler particles exposed on the surface of the fibers, forming an uneven structure that allows cells to effectively attach to the surface of the fibers.

Fibers constituting non-woven mats produced by the method of one embodiment of the invention have excellent protein adsorption performance because the surface charged inorganic filler particles are exposed without being covered by a resin layer, and cells can adhere to the fiber surface via the proteins adsorbed on the surface of the fibers.

The non-woven mat produced by the method of an embodiment of the invention are formed by short fibers that are torn apart to become a number of short fibers having a length of 20 mm to 80 mm during the flight in the ES apparatus and depositing on the rotating drum collector. The fibers are aligned in a certain direction (see FIG. 3(a)). The alignment of the fibers in a constant direction is desirable for the adherent cells to migrate along the fibers.

The non-woven mat produced by the method of an embodiment of the invention is formed by a process in which a number of short fibers of 2-30 mm in length that are torn apart during the flight in the ES apparatus is deposited on the rotating drum collector such that a number of curved short fibers are deposited in random directions to form a mesh structure (see FIG. 3(B)). The mesh structure has an excellent ability to trap seeded MSCs in the mat.

The non-woven mat produced by the method of an embodiment of the present invention is flexible in three-dimensional directions and is not broken when bending pressure is applied to the non-woven mat, and thus handling of the mat is easy.

The non-woven mat produced by the method of an embodiment of the present invention is made of 2 mm to 80 mm curved short fibers that are deposited and intertwined to form a 0.1 mm to 0.5 mm thickness, forming an excellent three-dimensional culture substrate.

The specific surface area of the fiber constituting the non-woven mat produced using the method of an embodiment of the present invention is significantly increased due to the formation of countless number of bubble pores with diameters of around 1 μm or less throughout the entire surface of the fiber (see FIGS. 5(a) and 5(b)). In addition, a physical uneven structure is formed on the fiber surface by the countless number of bubble pores and the countless number of filler particles exposed on the fiber surface, which allows the cells to be trapped in the non-woven mat and attach to the fiber.

The fibers that form the non-woven mats used as a cell culture substrate of the present invention are biocompatible and biodegradable, allowing the mat having adhesively cultured cells to be transplanted directly into the patient's body.

The non-woven mat used as a cell culture substrate of the present invention can be manufactured inexpensively and efficiently using the electrospinning method similarly to ReBOSSIS (registered trademark), allowing MSCs to proliferate on a commercial basis.

### BRIEF DESCRIPTION OF THE DRAWING

FIG. 1 shows an ES apparatus that is used in an embodiment of the invention.

FIG. 2(A) shows an external view of HAp50 mat, which is an embodiment of the invention, and FIG. 2(B) shows an external view of HAp70 mat, which is an embodiment of the invention.

FIG. 3(A) shows a SEM photograph (30×) of the PLGA fibers that form the HAp50 mat of the invention, and FIG. 3(B) shows a SEM photograph (30×) of the PLGA fibers that form the HAp70 mat of the invention.

FIG. 4(A) shows an SEM photograph (200×) of the PLGA fibers that form the HAp50 mat of the invention, and FIG. 4(B) shows an SEM photograph (200×) of the PLGA fibers that form the HAp70 mat of the invention.

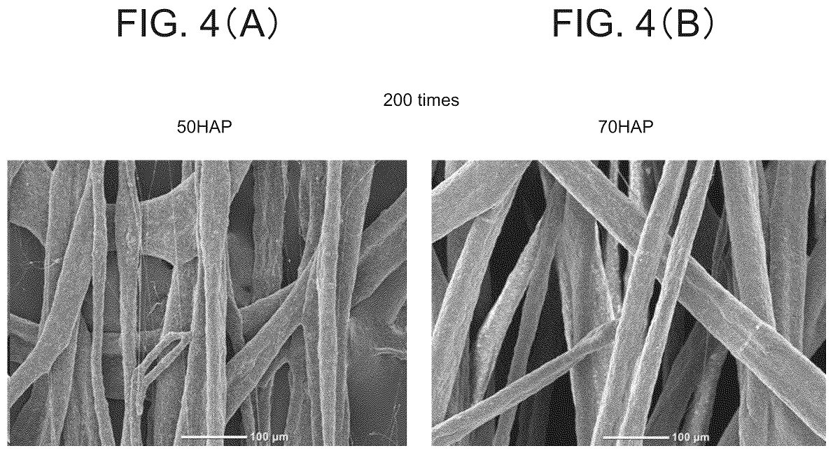

FIG. 5(A) shows an SEM photograph (1000×) of the PLGA fibers that form the HAp50 mat of the invention, and FIG. 5(B) shows an SEM photograph (1000×) of the PLGA fibers that form the HAp70 mat of the invention.

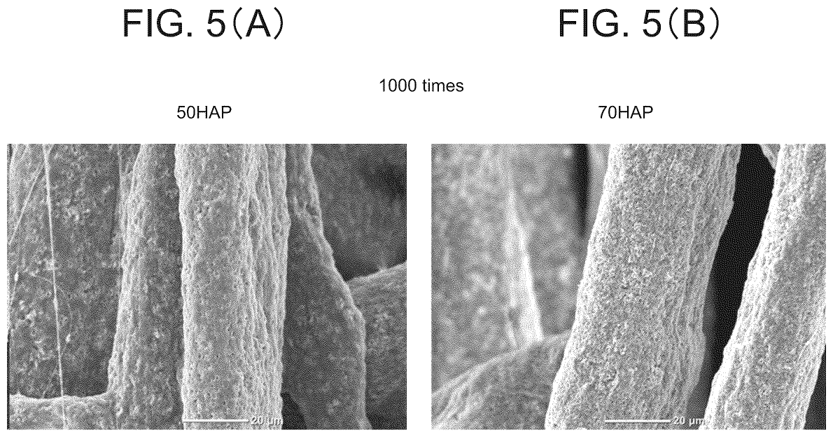

FIG. 6 shows a result of a protein (BSA) adsorption experiment on a non-woven mat of the invention.

FIG. 7 shows a result of protein (fibronectin) adsorption experiments on the non-woven mat.

FIGS. 8 (A) and (B) show a result of the MSC culture experiment using serum-free medium containing fibronectin.

FIGS. 9 (A) and (B) show a SEM photograph of needle-shaped HAp particles used in the embodiment of the invention.

FIGS. 10(A) and 10(B) show the results of HCL immersion experiments conducted to confirm that HAp particles exposed on the fiber are not covered by a thin resin layer.

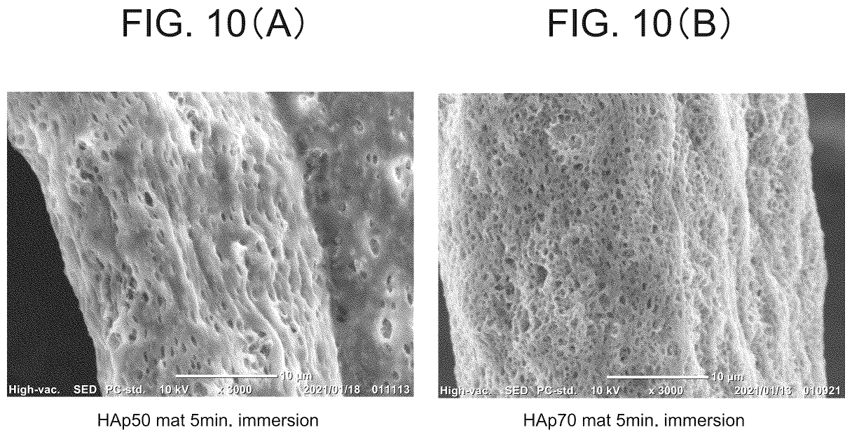

Hereinafter, preferable embodiments of the present invention are explained by referring to the drawings.

### (1) Materials Used in the Invention

### <Biocompatible Resin>

As the resin that is fibrillated by using electrospinning method, biocompatible resins such as PLA, PLGA, and PCL can be used as long as they can be dissolved by a solvent. Because biocompatible resin such as PLA, PLGA, and PCL have hydrophobic group, those resins are suitable for the adsorption of hydrophobic portions of proteins. Because PLGA is an amorphous resin, it is suitable for the preparation of a composite by using a kneading process.

When PLGA is used, molecular weight of the resin should be between 150,000 and 400,000 to produce a fiber from the resin using electrospinning method. More preferably, 200,000 to 400,000 is preferred. If the molecular weight is lower than 150,000, entanglement of molecular chains becomes weak, and the resin may not maintain its shape as a fiber. Conversely, if the molecular weight is greater than 400,000, viscosity of the spinning solution becomes too high, and as a result, it becomes necessary to decrease the resin concentration by increasing the proportion of solvent in the spinning solution to lower the viscosity. In that event, amount of solvent in the spinning solution becomes too large. As a result, it becomes difficult to sufficiently volatilize the spinning solution during flight after the spinning solution is ejected from the nozzle, resulting in a difficulty for the spinning solution to become fibers.

### <Inorganic Filler Particles>

Inorganic filler particles used in the present invention are sized such that the particles can be uniformly dispersed in the ES spinning solution and form an uneven structure on the surface of the fiber where the particles are exposed so that cells can easily attach to the fiber. In a preferred embodiment of the invention, particles of 1 μm to 5 μm size are used.

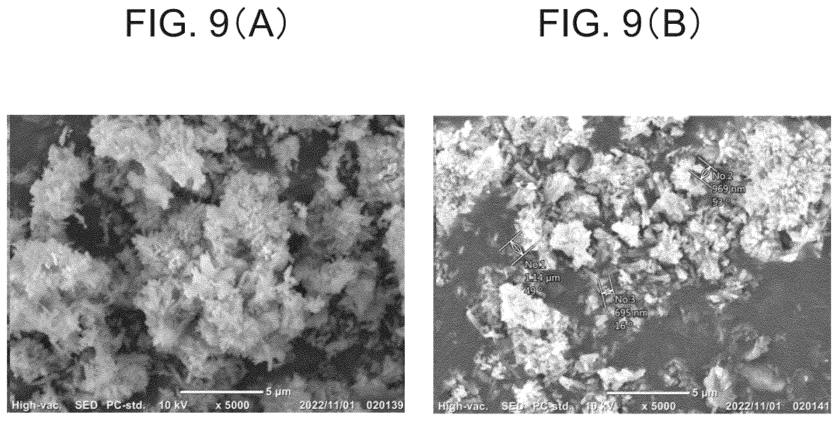

FIG. 9 shows a SEM photograph of needle-shaped HAp particles. The diameter of the needle-shaped HAp is around 10 μm when purchased from the manufacturer. However, during the kneading process of an embodiment of the invention, the particles are finely ground and becomes to 1 μm to 5 μm.

ReBOSSIS (registered trademark) uses bioabsorbable β-TCP particles as a filler because it is implanted in the body. However, bioabsorbability is not necessary for a cell culture substrate that is used in vitro. HAp particles are not bioabsorbable but have positively charged crystal face and negatively charged crystal face in a neutral PH environment. The HAp particles have both positively and negatively charged crystal faces in a neutral PH environment, which gives them excellent protein adsorption performance. Needle-shaped HAp particles are particularly desirable because they have a positively charged a-face in the longitudinal direction of the needle and can effectively adsorb proteins having a negative effective surface charge (adhesive proteins) in a neutral PH environment.

### <Volatile Solvent>

The solvent that is used for the method of present invention is preferably a material that can dissolve a solvent soluble resin, and is highly volatile with a boiling point below 200° C. under atmospheric pressure conditions and are liquid at room temperature. Chloroform is preferred as a volatile solvent for use in the method of the present invention because it has excellent solubility of biocompatible resins and high volatility.

### (2) Configuration of the Electrospinning Apparatus Used in the Invention

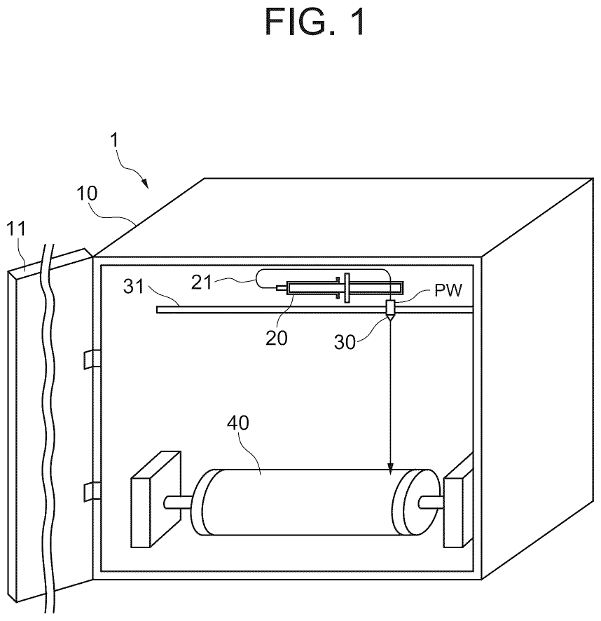

FIG. 1 shows the configuration of the electrospinning apparatus used in the present invention. In FIG. 1, electrospinning apparatus 1 has a housing 10 that is equipped with a syringe 20, a nozzle 30, and a rotating drum 40. Housing 10 is preferably made of conductive material such as steel to avoid electrostatic charge. Rotating drum 40 is electrically grounded.

### <Housing>

Housing 10 can be shut off from the outside air by closing the front door 11, which is mounted so that the front opening can be opened and closed and the temperature and humidity inside the housing can be adjusted according to individual spinning conditions. The housing 10 is equipped with an exhaust fan to allow ventilation while the equipment is in operation. Rail 31 is mounted near the ceiling of the housing 10, which is a facility for sliding the nozzle 30 in the horizontal direction. In the method of the present invention, temperature and humidity in the housing should be adjusted to 15° C. to 30° C. and 50% or less in order for the spinning solution emitted from the nozzle to deposit on the rotating drum in the form of fibers, since the solvent need to evaporate sufficiently while the emitted fibers are falling and flying inside the housing 10.

### <Syringe>

Syringe 20 is fixed near the ceiling of housing 10. Syringe 20 may be mounted on rails provided in housing 10, and syringe 20 itself may be configured to slide along with nozzle 30 on the rail.

After the syringe 20 is filled with the spinning solution, the switch to start spinning is pressed, and then the spinning solution filled in the syringe 20 is pushed through the tube to the nozzle 30 at a constant pressure/rate to start the electrospinning process. In one embodiment of the invention, the amount of spinning solution that can be filled into the syringe is set to be 10 ml.

### <Nozzle>

In FIG. 1, nozzle 30 is connected to syringe 20 via tube 21. Nozzle 30 is installed so that it can slide on rails in the housing 10. Syringe 20 may be fixed to the housing 10. Nozzle 30 that is connected to the syringe 20 via flexible tube 21 may be configured to move horizontally on the rail 31 (to the extent that the tube 21 can reach). Nozzle 30 has a hollow needle made of conductive material, and the spinning solution extruded from the syringe 20 is introduced into the nozzle and discharged from the tip of the needle.

### <Nozzle Diameter>

Nozzles that can be used in the electrospinning equipment of the present invention are 27 G, 22 G, and 18 G depending on the size of diameter. Sizes of diameter of 27 G, 22 G, and 18 G are shown in the following table.

| Name | Outer diameter (mm) | Inner diameter (mm) |
|---|---|---|
| 27G | 0.41 ± 0.02 | 0.22 ± 0.03 |
| 22G | 0.72 ± 0.02 | 0.41 ± 0.03 |
| 18G | 1.25 ± 0.02 | 0.82 ± 0.03 |

### <Power Supply>

A DC power supply (PW) having adjustable voltage is connected to nozzle 30. When the DC power supply is turned on, a positive high voltage is applied to nozzle 30, making nozzle 30 a positive electrode. Rotating drum 40, which is electrically grounded, becomes a negative electrode by electrostatic induction, generating an electric field between nozzle 30 and rotating drum 40. At the tip of nozzle 30, positively charged spinning solution discharged from the nozzle is subjected to electrostatic attraction, causing the Taylor cone phenomenon, and is ejected into the air in a form of fibers.

### <Rotation Drum>

At the bottom of housing 10, rotating drum 40 is installed to collect the fibers produced by electrospinning as a non-woven mat. Rotating drum 30 is electrically grounded. When a positive voltage is applied to nozzle 30, electrostatic induction occurs, and the rotating drum 40 is negatively charged and becomes the opposite electrode of the nozzle 30.

The rotating drum is wound with a conductive aluminum sheet or a highly peelable sheet such as silicone sheet. The fibers emitted from the nozzle 30 and flying down can be wound onto the drum around a rotating take-up shaft to obtain a non-woven mat. At this time, the fibers deposited on the drum are charged by the high voltage of ES, so they repel each other on the surface of the drum where they are deposited. When the drum rotation speed is fast, the fibers have a strong tendency to align and orient in the winding direction, but when the drum rotation speed is slow, the fibers' repulsive force against each other prevails, and as a result, the fibers have a strong tendency to orient in a random direction.

### (3) Electrospinning of the Invention

### <Preparation of Spinning Solution>

The biocompatible resin and inorganic filler particles are mixed and kneaded in a kneader to produce a composite, and the composite is dissolved in a solvent to obtain a spinning solution. In the present invention, the composition of the biocompatible fibers constituting the non-woven mat is determined by the mixing ratio of the biocompatible resin and inorganic filler particles in the composite prepared by kneader kneading. For example, HAp50 mat is a non-woven mat composed of biocompatible fibers containing 50% by weight (24.2 vol %) of HAp particles and 50% by weight (75.8 vol %) of biocompatible resin. HAp70 mat is a non-woven mat composed of 70% by weight (42.6 vol %) of HAp particles and 30% by weight (57.4 vol %) of biocompatible resin.

In the present invention, a composite with a uniform dispersion of calcium salts in an amount exceeding 20 vol % (about 45 wt %) can be prepared by using the kneading method. A spinning solution having a large amount of inorganic particles uniformly dispersed can be prepared by dissolving the composite thus prepared in a solvent such as chloroform. Details of the kneading method are described in PCT/JP2017/016931 (WO2017/188435). Resin concentration of the spinning solution must be below a certain level to be able to smoothly deliver the spinning solution from the syringe to the nozzle. On the other hand, resin concentration of the spinning solution must be above a certain level in order for the resin to act as a binder for the filler particles to form continuous fibers.

True density of calcium compound particles is higher than that of PLGA. For example, PLGA has a density of 1.01 g/cm³, HAp has a density of 3.17 g/cm³, and β-TCP has a density of 3.14 g/cm³. Therefore, wt % and vol % correlate as follows:

TABLE 1
HAp content correlations

| | | | | | | | | | |
|---|---|---|---|---|---|---|---|---|---|
| wt % | 90 | 80 | 70 | 60 | 50 | 40 | 30 | 20 | 10 |
| vol % | 74.1 | 56.0 | 42.6 | 32.3 | 24.2 | 17.5 | 12.0 | 7.4 | 3.4 |

TABLE 2
β-TCP content correlations

| | | | | | | | | | |
|---|---|---|---|---|---|---|---|---|---|
| wt % | 90 | 80 | 70 | 60 | 50 | 40 | 30 | 20 | 10 |
| vol % | 74.3 | 56.3 | 42.9 | 32.5 | 24.3 | 17.7 | 12.1 | 7.4 | 3.5 |

In ReBOSSIS (registered trademark), composition of calcium salts (bone formation factor) in the fibers comprising the cotton shape is controlled by weight percent. However, since in the present invention it is important that the large amount of inorganic filler particles are physically exposed on the surface of the fiber to form an uneven structure to which cells can easily attach, it is reasonable that the amount of inorganic filler particles contained in the fiber is determined by volume that the particles occupy in the fiber, not by weight percentage. Therefore, in the present invention, the amount of inorganic filler particles is indicated by converting weight percentages to volume percentages based on the content correlation table above.

### <Feeding Spinning Solution to the Nozzle>

In the method of the present invention, the spinning solution filled in the syringe is delivered at a faster rate than normal ES. As a result, the flow rate per second of the spinning solution at the nozzle outlet becomes larger. In an embodiment of present invention, when the spinning solution filled in the syringe is fed at a rate of 3 ml/h to 15 ml/h to the discharge port having an inner diameter of 0.4 mm to 1.0 mm, flow rate of the spinning solution by volume at the nozzle outlet is 0.83 mm³/sec to 4.2 mm³/sec, mass flow rate is 1.2 mg/sec to 6.8 mg/sec. As the volume per unit time of the spinning solution extruded from the nozzle outlet increases, the force that causes the spinning solution ejected from the nozzle to fall increases under the action of gravity due to its own weight. As a result, the degree of swinging of flight trajectory of the emitted spinning solution is decreased because repulsive force due to the bias of the electric charge becomes smaller compared to the case where the spinning solution is pulled down to the drum collector only due to the pulling force of the electric field.

### <Application of Voltage>

Upon filling the spinning solution into the syringe, DC power supply is turned on and voltage is applied to the nozzle 30. By applying voltage to the nozzle 30, the filled spinning solution is charged and a potential difference is created between the installed drum collector and the nozzle, causing the charged spinning solution to be pulled toward the drum by the Taylor cone phenomenon.

### (4) Non-Woven Mat is Formed on Rotating Drum Collector

### <Formation of Short Fibers>

In the method of present invention, the spinning solution prepared using the kneading method contains a large amount of inorganic filler particles. When the spinning solution is ejected from the nozzle in the form of fibers, the inorganic filler particles are held together with resin as a binder, and form longitudinally continuous fibers in that state. However, if the flight trajectory is violently shaken by the repulsive force caused by the bias of the electric charge during the flight in the ES apparatus, the filler particles becomes unable to maintain the state of being held together by the resin, and the fibers are torn off during the flight process, resulting in formation of a number of short fibers, which are then deposited on the rotating drum collector.

In the method of the present invention, the spinning solution filled in the syringe is fed at a high feed rate to the discharge port of the nozzle having a large diameter, so a large amount of spinning solution is pushed downward from the discharge port per unit time, and the extruded spinning solution falls downward by gravity. At the same time, the solution is pulled toward the collector by the force of the electric field generated between the nozzle and the collector by applying high voltage to the nozzle. The pulling force due to the electric field is subjected to a repulsive force caused by the bias of the electric charge, and the flight trajectory swings. However, reduction of fiber diameter caused by the oscillation of fiber flight path which occur in the bending instability phenomenon of usual electrospinning does not occur. Only reduction of fiber diameter that is caused by evaporation of solvent and swinging of flight trajectory occurs.

In order for the phenomenon of swinging flight trajectory of the present invention to occur, the electric field formed between the nozzle and the rotating drum must be above a certain degree. The strength of the electric field is determined by the value of the applied voltage and the distance between the nozzle 30 and the rotating drum 40 under the formula V=Ed. Therefore, the value of the applied voltage required to generate the swinging of flight trajectory phenomenon cannot be determined alone. However, the value of the applied voltage is necessarily set higher than a certain value because the flight distance of the fiber emitted from the nozzle must be greater than a certain distance because the solvent must be evaporated during that time of flight. In one example of this invention, the distance between the nozzle and the drum is 200 mm, and the voltage applied to the nozzle is set to be 28 kV.

By reciprocating the spinneret horizontally for 1 hour at a moving speed of 2 cm/s with a moving width of 10 cm while spinning fibers from the nozzle, the short fibers are distributed and deposited on the rotating drum collector, and the fibers adhere and bond to each other in that state to form a non-woven mat. Thickness of the non-woven mat should be about 0.1 mm to 0.5 mm in order to seed MSCs over the entire area of the mat while maintaining the three-dimensional structure required for a three-dimensional cell culture substrate, and to detach and collect the cells that have been seeded, attached to the mat, and proliferated from the mat.

### (5) Collection of Non-Woven Mat

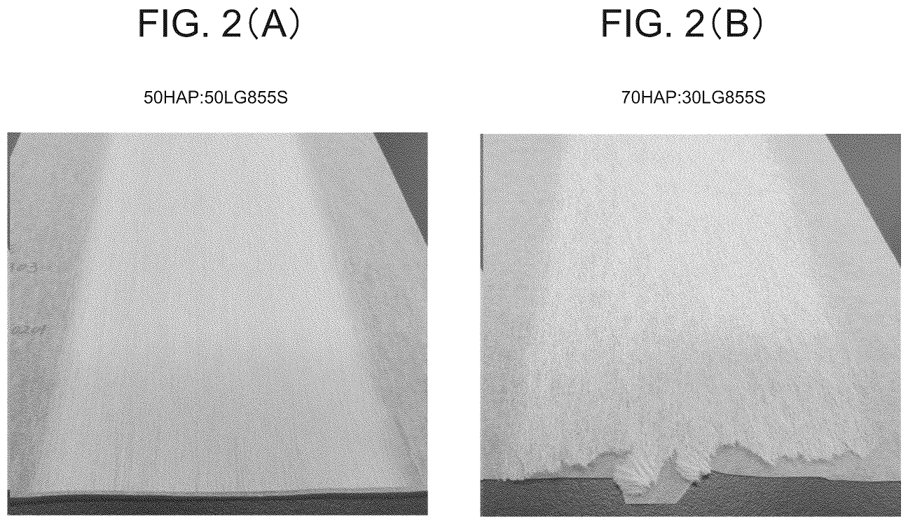

After depositing the fibers as a non-woven mat on a rotating drum that is wound with a peelable sheet such as aluminum or silicone sheet, the mat can be collected by removing the aluminum sheet from the rotating drum. In the method of the present invention, multiple short fibers reach the drum in a winding state as they are shaken out of their trajectory during the falling flight. Thus, the multiple short fibers, which are wound and curved, adhere and bond with each other on the drum to form a non-woven mat. FIG. 2 shows an external view of the non-woven mat. FIG. 2(A) shows a non-woven mat made of fibers containing 50% HAp by weight, and FIG. 2(B) shows a non-woven mat made of fibers containing 70% HAp by weight.

### (6) Properties of Fibers Constituting the Non-Woven Mat of the Invention

### <Bubble Pore Size of Fiber>

The fiber produced by the method of the present invention has countless number of bubble pores of around 1 μm or smaller diameter throughout the entire surface of the fiber. The mechanism by which such bubble pores are formed is that the fiber emitted from the nozzle contains a large amount of chloroform, and the chloroform contained in the fiber evaporates as bubbles inside and on the surface of the fiber during flight. When the bubbles generated inside of the fiber go out, several bubbles combine to reduce the surface area and become larger bubbles that go out of the fiber. Bubbles generated near the surface of the fiber are smaller than those generated inside the fiber. Fibers emitted from the nozzle are constantly exposed to fresh air as they fly through the air and continue to receive thermal energy, which accelerates vaporization near the surface. By receiving a lot of energy, a large number of bubbles are generated, and it is considered that some of them combine to reduce the surface area, causing the bubbles to become larger.

Size of the bubble pores formed on the surface of fibers spun by this method is determined by the viscosity of the polymer. In an embodiment of the present invention, diameter of the bubble pores formed by the air bubbles breaking out was 0.1 to 3 μm. To form bubble pores on the surface of the resin fibers, it is effective to blow air in the ES apparatus.

### <Protein Adsorption Performance>

The fibers that form the non-woven mat of the present invention have the ability to adsorb adhesive proteins contained in the culture medium on the surface of the fibers.

As for the mechanism by which proteins are adsorbed on the surface of substrates, it is known that proteins are adsorbed on the surface of polymer materials through hydrophobic interactions originating from the protein molecules themselves. Proteins are high polymers consisting of amino acids with hydrophilic and hydrophobic groups, and the surfaces of protein molecules have a mosaic structure consisting of hydrophilic and hydrophobic portions. Resins such as PLA, PLGA, and PCL can be used suitably because they have hydrophobic groups.

Furthermore, amino acids that constitute the protein molecule have both amino groups and carboxyl groups, and thus have an isoelectric point. Acidic amino acids having multiple carboxyl groups have a lower isoelectric point, while basic amino acids having multiple amino groups have a higher isoelectric point. Therefore, in a neutral PH solution, acidic proteins tend to be negatively charged and basic proteins tend to be positively charged. Ceramic particles have high surface energy and their surfaces are positively or negatively charged. Therefore, when ceramic particles are immersed in a neutral PH medium containing proteins, acidic proteins are more likely to be adsorbed on particles with positively charged surfaces because their effective surface charge is negative in a neutral PH environment, and basic proteins are more likely to be adsorbed on particles with negatively charged surfaces because their effective surface charge is positive.

Protein adsorption based on electrostatic interactions is more adsorptive than that based on hydrophobic interaction. Therefore, in order to obtain high initial cell attachment to the scaffold material, the surface of the fiber should be charged opposite to the effective surface charge of the protein. HAp can be suitably used because the Ca2+ present on the particle surface adsorbs acidic proteins having negative effective surface charge under neutral pH.

Fibers produced by using the electrospinning method of the present invention have inorganic filler particles exposed on the fiber surface to form an uneven structure thereon, but it is possible that the exposed particles are covered by a thin resin layer. Since the surface charge of inorganic particles is weak, if the surface of the particles is covered by a resin layer, even if it is a very thin layer, the ability of the particles to adsorb proteins is lost. Based on this consideration, thinking of the fact that HCL dissolves HAp but not resin (PLGA), the inventors of this invention conducted an experiment in which fibers containing HAp particles were immersed in HCL solution and observed whether the fibers were covered by resin by observing whether HAp was dissolved or not.

FIG. 11 shows the result of immersing (A) HAp50 mat and (B) HAp70 mat in HCL solution for 5 minutes and observing the changes on the surface shape of the fibers. The result of the experiment showed that more vacancies were formed on the fiber surface of the fibers of the HAp70 mat than those of the HAp50 mat. This is a result of that greater amount of HAp particles are exposed on the surface of the fibers of the HAp70 mat, which were dissolved by the HCL. If the calcium phosphate particles were covered by a PLGA resin layer on the fiber surface, this would not have been the case because the presence of the resin layer, which is insoluble in HCL, would have prevented the HAp particles from contacting the HCL and thus dissolving the particles. The result of this experiment confirmed that the inorganic filler particles exposed on the surface of fibers produced by using the electrospinning method are not covered by a thin resin layer, and that more HAp particles are exposed on the fiber surface in the HAp70 mat than in the HAp50 mat.

### EXPERIMENT

### I. Experiment of Preparing a Non-Woven Mat

Experiment 1 in which experimental sample of non-woven mat (HAp50 mat) consisting of fibers produced by electrospinning using a spinning solution prepared by preparing a composite of 50 wt % (24.2 vol %) of HAp particles and 50 wt % (75.8 vol %) of PLGA resin by kneading and dissolving it in chloroform and Experiment 2 in which a sample of non-woven mat (HAp70 mat) consisting of fibers produced by the electrospinning method using a spinning solution prepared by preparing a composite of 70 wt % (42.6 vol %) HAp particles and 30 wt % (57.4 vol %) PLGA resin using the same kneading method, and dissolving the composite in chloroform were conducted.

### Experiment 1

<HAp50 mat>
Composition: Needle-shaped HAp 50 wt % (24.2 vol %)/PLGA 50 wt % (75.8 vol %)
Feeding rate: 10 ml/hr
Resin concentration: 9%
Flight distance: 200 mm
Temperature in ES apparatus: 29° C. Humidity in ES apparatus: 47%
Fiber diameter: 10 to 80 μm
Fiber length: approx. 5 cm

### <Outline of Experiment and Result>

The spinning solution (resin concentration 9%) containing 50 wt % HAp particles as filler is ejected from the nozzle in a fiber-like shape in downward direction at a feeding rate of 10 ml/hr and a voltage of 28 kv.
The spinning solution (chloroform+filler+resin) emitted from the nozzle evaporates chloroform during the flight process to form fibers. However, because the filler component is 50 wt % and the resin component is small, it does not form a long continuous fiber.
If the resin concentration is less than 7%, there is not enough resin to become a binder for the filler particles, so the fibers will not be continuous. If the resin concentration is 11% or higher, the viscosity of the spinning solution is too high, making it difficult to pass the spinning solution through the nozzle.
The spinning solution emitted from the nozzle contains an excess of chloroform. The chloroform evaporates in the process of being pulled toward the drum by the electric field as it flies. However, the chloroform is still abundant when it reaches the drum. The chloroform remaining in the fibers after the fibrous spinning solution is deposited on the drum evaporates there over time, and the evaporation of chloroform from the fibers solidifies the fibers, thereby creating a non-woven fabric (FIG. 2(A)).
The fibers forming the non-woven fabric are oriented in one direction (oriented).

### Experiment 2

<HAP70 mat>
HAp70 wt %(42.6 vol %)/PLGA30 wt %(57.4 vol %)
Feeding rate: 5 ml/hr
Resin concentration: 10%.
Flight distance: 200 mm
Temperature in ES apparatus: 25.5° C. Humidity in ES apparatus: 25%
Fiber diameter: 40 to 70 μm
Fiber length: approx. 5 mm

### <Outline of Experiment and Result>

By applying a voltage of 28 kv at a feeding rate of 10 ml/hr, a spinning solution containing 10% resin concentration and 70 wt % filler was ejected from the nozzle in a fiber-like form in a downward direction.
The spinning solution (chloroform+filler+resin) emitted from the nozzle evaporates chloroform during the process of flight along an unstable trajectory and becomes fibers. However, because of the high filler content and low resin content, it was torn off to make a number of short fibers of less than 1 cm length, and it was difficult to form a single long continuous fiber.
When the resin concentration was less than 7%, the fibers did not become continuous because there was not enough resin to serve as a binder for the filler particles. When the resin concentration was 11% or higher, the viscosity of the spinning solution was too high, making it difficult to pass the spinning solution through the nozzle.
The spinning solution emitted from the nozzle is pulled by the electric field and deposited on the drum. Since the deposited fibers still contain abundant chloroform, the fibers easily adhered to each other at the points where they come into contact. The chloroform contained in the fibers evaporated from the fibers over time as the fibers adhered to each other at numerous points. The fibers deposited on the drum were further solidified in that state by the evaporation of chloroform to form a non-woven mat (FIG. 2(B)).

### Observation of the Result of Experiment 1 and Experiment 2

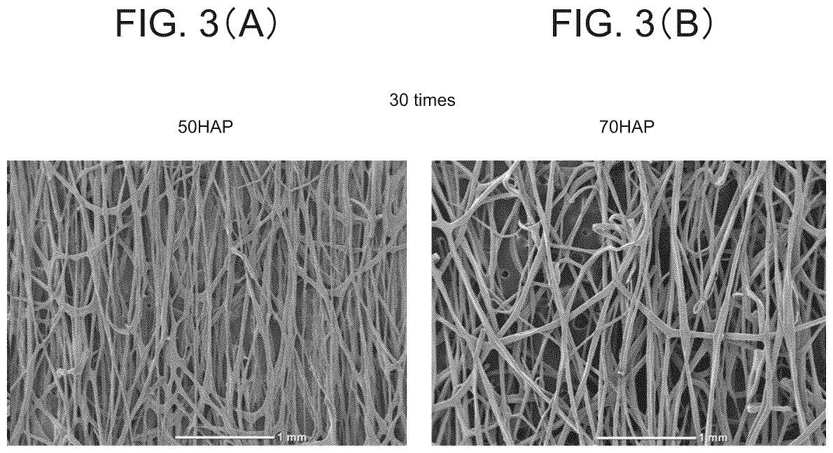

The length of the fibers constituting the non-woven mat differed significantly between the two cases where the amount of HAp is 50% by weight and 70% by weight. The former is around 5 cm, whereas the latter is about 1/10 of that length, around 5 mm. In the former case, the multiple fibers constituting the non-woven mat tended to be aligned and oriented in one direction (FIG. 3(A)), whereas in the latter, the fibers were randomly oriented and not aligned (FIG. 3(B)).
While both HAp50 mat and HAp70 mat can be used as cell culture substrates, HAp50 mat is considered superior to HAp70 mat because of the aligned fiber orientation for migration of cells attached to the non-woven mat.
The HAp50 mat was flexible and did not easily lose its mat shape when pulled or bent, whereas the HAp70 mat easily lost its mat shape by folding when bent and easily separated when pulled.
The result of Experiment 1 and Experiment 2 suggest that it is possible to adjust the amount of inorganic filler particles in the biocompatible fiber to an optimal composition between 50 wt % and 70 wt %.

### II. Protein Adsorption Experiment

Experiments were conducted to confirm the adsorption of proteins to the mat using serum albumin (BSA) and fibronectin, adhesive protein, as proteins to be adsorbed on the inorganic particles.

### (1) Immersion Experiment Using BSA-Containing Solution

(i) Place 500 μl of 10 μg/Ml BSA-containing solution in a 24-well plate.

### Method of Preparing BSA-Containing Solution:

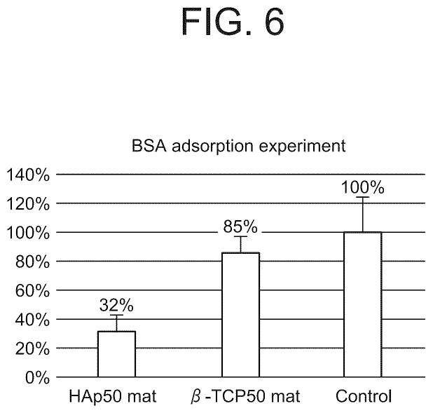

2 mg/ml of BSA solution: 2.5 μl
Purified water: 497.5 μl
   Above two were mixed to make a 200 times diluted BSA-containing solution. The PH of the solution was neutral.
(ii) A non-woven culture substrate made of ES fibers containing 50 wt % of needle-shaped HAp (HAp50 mat) and a non-woven culture substrate made of ES fibers containing 50 wt % β-TCP particles (β-TCP50 mat) were placed in 70% ethanol respectively. After washing with purified water, wipe off the water with a Kim wipe and immerse in a 24 well plate filled with BSA-containing solution. A well without non-woven mat immersed was also prepared and used as a control.
(iii) After an overnight still incubation at 37° C., the BSA concentration of each solution was measured in a well plate containing HAp50 mat, a well plate containing β-TCP50 mat, and a well plate without non-woven mat (control). The measurement result is shown in FIG. 6. In the result, concentration of BSA is shown by percentage, setting average fibronectin concentration (μg/ml) of the control as 100%.

### <Observation of Result of Experiment>

1) The concentration of BSA in both HAp50 mat and β-TCP50 mat decreased compared to the control. The decreased BSA is considered to be adsorbed on the cell culture substrates (HAp50 mat and β-TCP50 mat).
(2) For HAp50 mat, the content of BSA in solution was reduced to 32%, while for β-TCP50 mat, the content of BSA in solution was reduced to 86% only. The result of this experiment confirms that HAp50 mat have higher BSA adsorption performance than β-TCP50 mat.
(3) HAp50 mat have many HAp particles (50 wt %=about 25 vol %) exposed on the fiber surface without being covered by the resin layer, suggesting that the Ca2+ ions on the HAp particle surface adsorbed the acidic protein BSA (effective surface charge is negative in a neutral PH environment).

### (2) Immersion Experiment Using Fibronectin

(i) Place 500 μl of 10 μg/ml fibronectin-containing solution in 24-well plate.

### Preparation of a Solution Containing Fibronectin:

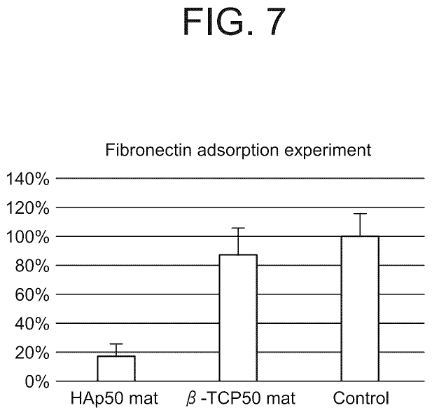

fibronectin human plasma (1 mg/ml): 5 μl
Purified water: 495 μl
   Above two were mixed to make a 100 times diluted solution containing fibronectin. PH of the solution was neutral.
(ii) A non-woven culture substrate made of ES fibers containing 50 wt % of needle-shaped HAp (HAp50 mat) and a non-woven culture substrate made of ES fibers containing 50 wt % of β-TCP particles (β-TCP50 mat) are placed in 70% ethanol, respectively, After washing with purified water, wipe off the water with a Kim wipe and immerse in 24-well plate filled with fibronectin-containing solution. A well without immersion of non-woven mat was also prepared and used as a control.
(iii) The solution was left still overnight at 37° C., and the fibronectin concentration of each solution was measured in a well plate with HAp50 mat, a well plate with β-TCP50 mat, and a well plate without mat (control). Result of the experiment is shown in FIG. 7. In the result, concentration of fibronectin is shown by percentage, setting average fibronectin concentration (μg/ml) of the control as 100%.

### Observation of Result of Experiment

1) Both HAp50 mat and β-TCP50 mat decreased the concentration of fibronectin in solution compared to the control. The decreased fibronectin is thought to have been adsorbed onto the cell culture substrate.
(2) The HAp50 mat reduced the fibronectin content in solution to 19%, while the β-TCP50 mat reduced the fibronectin content in solution to 82% only. These results confirm that HAp50 mat has a higher fibronectin adsorption performance than β-TCP50 mat.
(3) In the HAp50 mat, many HAp particles (50 wt %=about 25 vol %) are exposed on the fiber surface without being covered by the resin layer, suggesting that Ca₂⁺ ions on the HAp particle surface adsorbed the acidic protein fibronectin (effective surface charge is negative in a neutral environment). The surface of the HAp particles is exposed without being covered by a resin layer.

### III. MSC Proliferation Experiment

Non-woven mat samples formed of PLGA resin fibers produced by electrospinning method: a sample containing 50 wt % HAp and a sample containing 50 wt % β-TCP were prepared. Each sample was immersed in serum-free medium containing adhesion protein (fibronectin). Suspension of MSC (ADSC Lonza) suspensions were seeded. Attachment and proliferation of the cells of each sample was observed and compared.

### <Materials>

Cells: ADSC (MSC) Lonza
Medium: Cellartis TAKARA
Non-woven mat sample: HAp50 mat (PLGA:HAp=50:50) by weight
   β-TCP 50 mat (PLGA: β-TCP=50:50) by weight
   Dimension size of mat: ϕ=14 mm circular shape, 0.2 mm thick.
Reagents: Human plasma fibronectin 0.1% (1 mg/ml) SIGMA
CellTiter 96 (registered trademark) Non-Radioactive Cell Proliferation Assay (MTT) Promega
   Dye Solution
   Stop Mix
Accumax (release agent): Nakaraitesk

### Procedure of Experiment

### Step 1: Preparation of Cell Suspension

Fibronectin reagent was added at a rate of 1 M 1 to 1 ml of serum-free medium to make F medium of a concentration of 1 μg/ml.
ADSCs in cryopreservation were grown in culture after thawing, and the grown cells were used as ADSCs for the culture test.
The grown ADSCs were detached and diluted to 50,000 cells/500 μl using F medium to make a cell suspension.

### Step 2: Pre-Treatment of Non-Woven Mat Sample

Each non-woven mat SAMPLES were treated by immersing them in an amphiphilic 70% ethanol and serum-free medium solution so that they would not repel immersion in water and medium, and then used in 24 well plates.

### Step 3: Cell Seeding

Cell suspension (50,000 cells/500 μl) was seeded dropwise onto each non-woven mat sample in a 24 well plate.

### Step 4: Cell Count

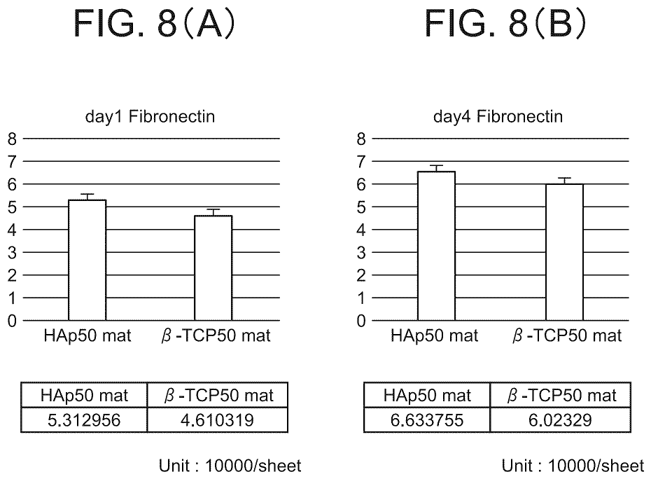

The number of cells that attached and proliferated on each non-woven mat sample was measured by MTT assay and absorbance analysis. The measurement results are shown in FIGS. 8(A) and 8(B).

### Observation of Result of Experiment

(1) After 1 day of culture, the number of cells attaching to the non-woven mat SAMPLES was measured to be about 53,000 for the HAp50 mat and 46,000 for the β-TCP50 mat (FIG. 8(A)). This result may be due to the small number of cells in the suspension (50,000 cells/500 μl) that were not captured by the non-woven mat sample and settled to the bottom of the well plate, and the structure of the HAp50 mat and β-TCP50 mat was suitable for cell attachment. The reason for this may be that the structure of the HAp50 and β-TCP50 mats was suitable for cell attachment.
2) HAp50 mat showed better initial adhesion of cells than β-TCP50 mat, and subsequent growth was also better than β-TCP50 mat. This result may be due to the effective adsorption of fibronectin, used as an adhesion protein, on the fiber surface of HAp50 mat.
(3) After 4 days of culture, the number of cells adhering to the non-woven mat sample was measured to be about 65,000 for HAp50 mat and 60,000 for β-TCP50 mat (FIG. 8(B)). It is considered that cells adhering to the non-woven mat on the first day of culture grew steadily on the non-woven mat for the next three days.

Since the non-woven mat is formed of biocompatible and biodegradable fibers, it can be transplanted into a living body with cells attaching to it. The fact that such high cell attachment and proliferation could be achieved in the present invention while using such cell culture substrates was a groundbreaking achievement in this technical field.

The present invention has been described above in the example of a non-woven mat sheet made of fibers produced by using electrospinning method. However, the sheet is not limited to a non-woven mat as long as it can be used as a cell culture substrate and has a structure that allows cells to penetrate and adhere to it.

### DESCRIPTION OF CODE

1 Electrospinning apparatus
10 Housing
11 Front door
20 Syringe
21 Tube
30 Nozzles
31 Rail
40 Rotating drum

1.-15. (canceled)

16. A method for culturing mesenchymal stem cells using a non-woven mat, the method comprising:
immersing a non-woven mat formed of biocompatible fibers in a medium that is filled in a cell culturing container, wherein:
the biocompatible fibers contain 20 to 50 vol % of HAp particles and 50 to 80 vol % of biocompatible resin;
a surface of the biocompatible fibers has an uneven structure formed by having HAp particles contained in the biocompatible fibers partially exposed thereon;
the non-woven mat has a three-dimensional structure in which a plurality of fibers with a fiber length of 2 mm to 80 mm and a fiber diameter of 10 to 80 m are entangled with each other and adhered and connected at multiple points of contact; and
adhesive proteins contained in the medium are adhered to the HAp particles that are exposed on the surface of the biocompatible fibers of the non-woven mat;
seeding a cell suspension containing a predetermined number of mesenchymal stem cells on the non-woven mat in the cell culturing container so that the mesenchymal stem cells are captured in the three-dimensional structure of the non-woven mat; and
leaving the non-woven mat still in a state that the mesenchymal stem cells are captured in the non-woven mat for a predetermined time period so that the mesenchymal stem cells are attached to the biocompatible fibers that are covered by the adhesive proteins adsorbed on the HAp particles and the mesenchymal stem cells attached to the biocompatible fibers are proliferated in the microenvironment formed in the three-dimensional structure of the non-woven mat.

17. The method of claim 16, wherein the adhesive protein contained in the medium has a negative effective surface charge and is adsorbed to the HAp particles that have a positive surface charge.

18. The method of claim 16, wherein the HAp particles contained in the biocompatible fibers are needle-shaped HAp particles.

19. The method of claim 16, wherein the medium is a serum-free medium containing an adhesive protein.

20. The method of claim 16, wherein the thickness of the non-woven mat is 0.1 mm to 0.5 mm.

21. A non-woven mat formed of biocompatible fibers for culturing mesenchymal stem cells, wherein:
the biocompatible fibers contain 20 to 50 vol % of HAp particles and 50 to 80 vol % of biocompatible resin;
a surface of the biocompatible fibers has an uneven structure formed by having HAp particles contained in the biocompatible fibers partially exposed thereon;
the non-woven mat has a three-dimensional structure in which a plurality of fibers with a fiber length of 2 mm to 80 mm and a fiber diameter of 10 to 80 m are entangled with each other and adhered and connected at multiple points of contact;
by immersing the non-woven mat in a medium containing adhesive protein, adhesive proteins contained in the medium are adhered to the HAp particles that are exposed on a surface of the biocompatible fibers of the non-woven mat; and
by seeding a cell suspension containing a predetermined number of mesenchymal stem cells on the non-woven mat in the cell culturing container, the mesenchymal stem cells are captured in the non-woven mat so that the mesenchymal stem cells are attached to the biocompatible fibers that are covered by the adhesive proteins adsorbed to the HAp particles and the mesenchymal stem cells attached to the biocompatible fibers are proliferated in the microenvironment formed in the three-dimensional structure of the non-woven mat.

22. The non-woven mat of claim 21, wherein the HAp particles contained in the biocompatible resin fibers are needle-shaped HAp particles.

23. The non-woven mat of claim 21, wherein the medium is a serum-free medium containing adhesive protein.

24. The non-woven mat of claim 21, wherein the thickness of the non-woven mat is 0.1 mm to 0.5 mm.
